(19) United States
(12) Patent Application Publication
(10) Pub. No.: US 2005/0182344 A1
(43) Pub. Date: Aug. 18, 2005

(54) Recovery belt apparatus

(76) Inventor: Pamela Dixon, Long Beach, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/781,085

(22) Filed: Feb. 17, 2004

(51) Int. Cl. A61F 5/00

(52) U.S. Cl. 602/1

(57) ABSTRACT

A device is presented including a pad portion attached to a first belt portion on a first end and a second belt portion on a second end. The device has a removable attaching means for attaching the first belt portion with the second belt portion. The pad portion is adapted to be placed over either a person's abdomen or thoracic area, and the removable attaching means adjusts pressure of the pad portion on either a person's abdomen or thoracic area.

### BACKGROUND

1. Field

This invention relates to post surgery/pregnancy recuperating devices, and more particularly to a comforting belt apparatus.

2. Description of the Related Art

In thoracic and abdominal chest surgical procedures, recovery in comfort can be a challenge. Different surgical procedures call for different location of incisions and orientation of the incision in reference to a person's abdomen or thoracic area. Thus, incisions can be in various areas and be slanted, horizontal or vertical.

One type of surgical procedure of the abdomen is a Cesarean section (C section) procedure for delivering a baby. A C section procedure is such that a baby is delivered through a surgical incision in the abdominal area of a woman. There are many reasons for a baby to be delivered by C Section, such as abnormal position of the baby (e.g., breech, transverse), macrosomia, cephalopelvic disproportion) or abnormalities of the placenta (e.g., placenta previa, placenta abruptio) and umbilical cord (e.g., prolapse). In these and other instances, vaginal birth is either not possible or not safe for the woman or child.

In typical (i.e., non-emergency) C section procedures, as illustrated in FIG. 1, surgical incision 10 is made substantially horizontal (i.e., a bikini cut) on an abdomen 15. In emergency C section procedures, as illustrated in FIG. 2, a substantially vertical incision 20 may be preferred, which allows quicker access to the baby. After the procedure is completed, the typical hospital stay is two to four days. The abdominal area will be sore and walking is encouraged to aid in healing.

One method to aid in pain management is to hold a pillow in place across the C section scar when coughing, sneezing, laughing or walking. Similar pain management strategies are advised for other surgical procedures of the thoracic and abdominal areas. The problem with this is that the pillow needs to be picked up or carried in order to use this method. For example, if a person is using a cane to assist them in walking, their other hand would need to hold a pillow (which probably doesn't allow them to hold anything else). In the case where a person may be using a walker, the person would need to stop and sit before being able to use their hands to hold a pillow. And, in the case where a woman may be holding her baby, the baby must be put down in order to un-occupy her hands.

### SUMMARY

One embodiment is presented including a pad portion attached to a first belt portion on a first end and a second belt portion on a second end and a removable attaching means for coupling the first belt portion with the second belt portion. The pad portion is adapted to fit to a person's abdomen and the removable coupling means adjusts pressure of the pad portion on a person's abdomen.

Another embodiment is presented including a pad portion having a first end, a second end, a third end and a fourth end. A first removable coupling means is included for attaching a first belt portion to one of the pad portion's ends. A second removable coupling means is included for attaching a second belt portion to another of the pad portion's ends. A third removable coupling means is included for attaching the first belt portion to the second belt portion. The pad portion is adapted to fit to a person's abdomen and the third removable coupling means adjusts pressure of the pad portion on a person's abdomen.

### BRIEF DESCRIPTION OF THE DRAWINGS

The embodiments are illustrated by way of example and not by way of limitation in the figures of the accompanying drawings in which like references indicate similar elements. It should be noted that references in the specification to “an embodiment,” “one embodiment,” “some embodiments,” or “other embodiments” means that a particular feature, structure, or characteristic described in connection with the embodiments is included in at least some embodiments, but not necessarily all embodiments, of the invention. The various appearances “an embodiment,” “one embodiment,” or “some embodiments,” are not necessarily all referring to the same embodiments. If the specification states a component, feature, structure, or characteristic “may”, “might”, or “could” be included, that particular component, feature, structure, or characteristic is not required to be included. If the specification or claim refers to “a” or “an” element, that does not mean there is only one of the element. If the specification or claims refer to “an additional” element, that does not preclude there being more than one of the additional element.

FIG. 1 illustrates a post Cesarean section (C section) horizontal scar on a person's abdomen.

FIG. 2 illustrates a post C section vertical scar on a person's abdomen.

FIG. 3A illustrates a first embodiment of a recovery apparatus adapted for horizontal placement on a person's abdomen.

FIG. 3B illustrates another embodiment of a recovery apparatus adapted for vertical placement on a person's abdomen.

FIG. 3C illustrates a lumbar support pad.

FIG. 3D illustrates another view of the lumbar support pad illustrated in FIG. 3C.

FIG. 4A illustrates a side view of the embodiment illustrated in FIG. 3A.

FIG. 4B illustrates a side view of the embodiment illustrated in FIG. 3B.

FIG. 5A illustrates the embodiment illustrated in FIG. 3A placed on a person's abdomen.

FIG. 5B illustrates the embodiment illustrated in FIG. 3B placed on a person's abdomen.

FIG. 6 illustrates still another embodiment of a recovery apparatus.

FIG. 7 illustrates a pad portion of the embodiment illustrated in FIG. 6 placed horizontally on a person's abdomen.

FIG. 8 illustrates the pad portion of the embodiment illustrated in FIG. 6 placed vertically on a person's abdomen.

FIG. 9 illustrates the lumbar support pad illustrated in FIGS. 3C-D placed on a person's lumbar area and the pad portion of the embodiment illustrated in FIG. 6 placed horizontally on a person's abdomen.

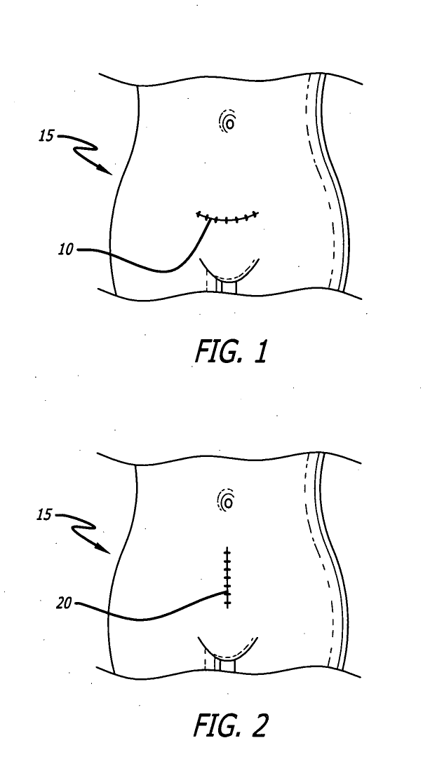

### DETAILED DESCRIPTION

The invention generally relates to a post surgery/pregnancy recovery apparatus. Referring to the figures, exemplary embodiments of the invention will now be described. The exemplary embodiments are provided to illustrate the invention and should not be construed as limiting the scope of the invention.

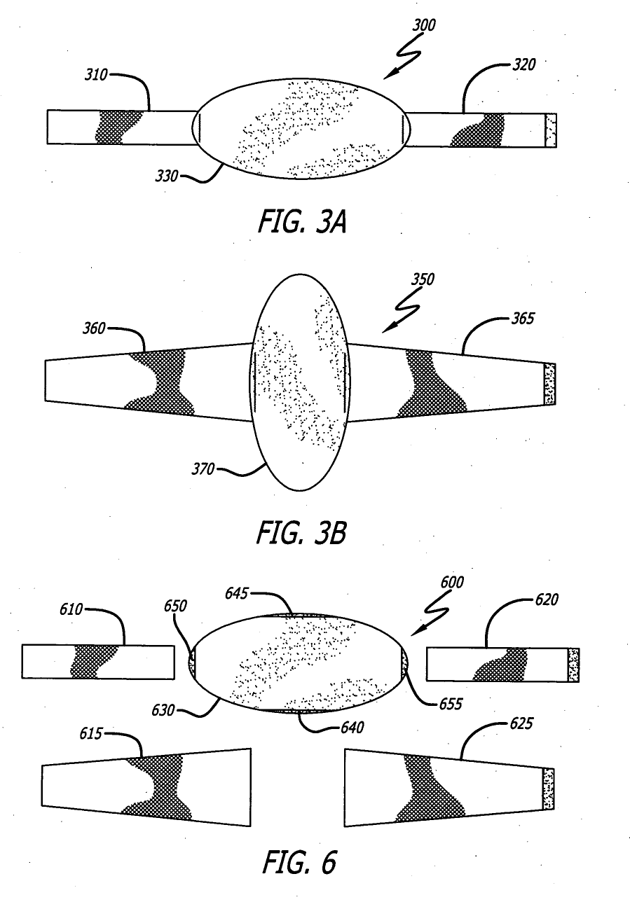

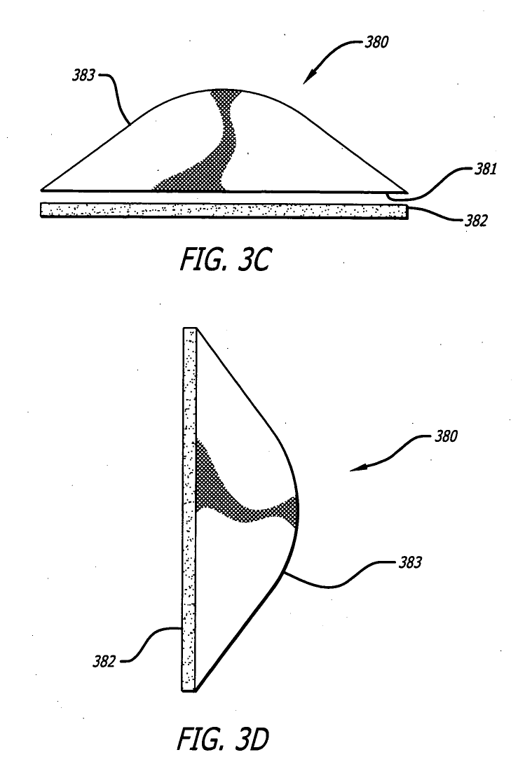

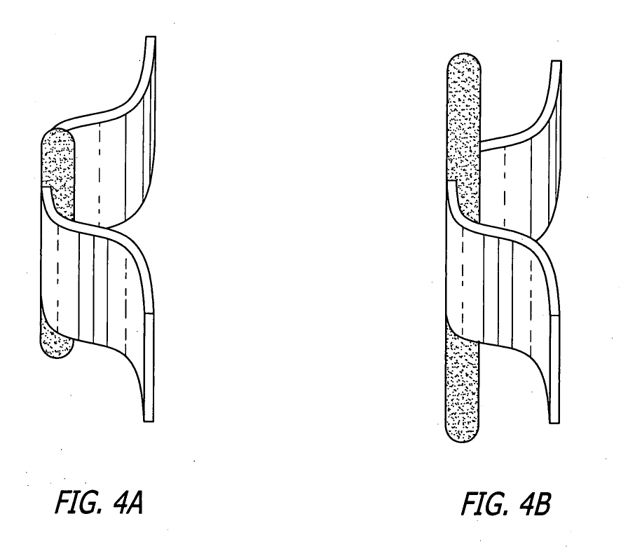

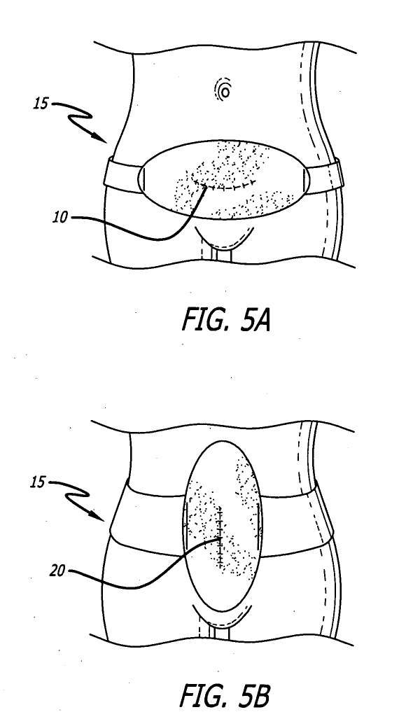

FIG. 3A illustrates an embodiment of a recovery belt apparatus. Recovery belt apparatus 300 includes pad portion 330 coupled to a first belt portion 310 on a first end 305 and a second belt portion 320 on a second end 307. This embodiment also includes a removable coupling means (not shown) for coupling first belt portion 310 with second belt portion 320. In this embodiment, pad portion 330 is adapted to fit to a person's abdomen and the removable coupling means adjusts pressure of pad portion 330 on a person's abdomen. Pad portion 330 is of such a size to fit a surgical area of a person typical of surgical procedures, such as Cesarean section (C section), laparoscopic hiatal hernia, hernia, tummy tuck, hysterectomy, appendectomy, gall bladder surgery, thoracic surgery (e.g., lung or heart surgery), etc. In one embodiment, pad portion 330 ranges from 9 inches to 18 inches in length, and 5 inches to 11 inches in height. It should be noted, however, that other lengths and widths can be used with recovery belt apparatus 300 as desired or necessitated. For example, for a larger person or for using as a post pregnancy abdominal shaper, recovery belt apparatus 300 would be desired to be larger e.g., 18″×11″, 11″×18″). FIG. 4A illustrates a side view of the embodiment illustrated in FIG. 3A. FIG. 5A illustrates the embodiment illustrated in FIG. 3A attached to a person's abdomen.

In one embodiment pad portion 330 is a pillow having a top portion and a bottom portion. The bottom portion makes contact with a person's abdomen. In another embodiment the bottom portion of the pillow has a concave shape. That is, the pillow is shaped to fit comfortably with a person's abdomen after a post surgery procedure. In yet another embodiment, pad 330 is shaped to fit comfortably to a person's thoracic area (i.e., curved appropriately to have continuous contact of pad 330 with a person's thoracic area. Pad 330 can be of a pillow thickness of 2 inches to 6 inches. It should be noted, however, that other thickness is possible, such as 8 inches, 9 inches, etc. In this embodiment, pad 330 is formable to a person's abdominal area to provide a comfortable fit when placed against. Pad portion 330 can be a foam rubber, down, latex, cotton, wool, polyester blends, fiber, poly-fill, etc. type of pillow.

In another embodiment, pad portion 330 is filled with gel, where the pad portion 330 (gel pad) has a top portion and a bottom portion that is adapted to fit to a person's abdominal area. The gel pad can contain thermoplastic elastomers (elastomeric materials), such as materials made from many polymeric families, including but not limited to the Kraton family of styrene-olefin-rubber block copolymers, thermoplastic polyurethanes, thermoplastic poly olefins, polyamides, polyureas, polyesters and other polymer materials that reversibly soften as a function of temperature. In one embodiment a Kraton block copolymer of styrene/ethylene-co-butylene/styrene or styrene/butadiene/styrene with mineral oil incorporated into the matrix as a plasticizer is used. In this embodiment, pad 330 is formable to a person's abdominal area to provide a comfortable fit when placed against. In other embodiments, pad portion 330 can contain a combination of gel and foam rubber. In yet another embodiment, the bottom portion of pad portion 330 is a gel pad with a concave shape adapted to fit a person's abdominal area where a surgical procedure has been performed.

In one embodiment, first belt portion 310 and second belt portion 320 are made of elastic material combinations (e.g., cotton/elastic/nylon, etc.), or other stretchable material (e.g., foam rubber, etc.) in order to vary total length of recovery belt apparatus 300, provide comfortable fit, provide flexibility, and also vary amount of pressure of pad portion 330 on a person's abdomen.

In one embodiment the removable coupling means (not shown) is a Velcro® type band disposed on inner and outer side surfaces of first belt portion 310 and second belt portion 320 for fastening the first and second belt portions together in an overlapping manner. That is, one of the belt portions has Velcro hooks, and the other has Velcro loops. When the hooks engage with the loops, the belt portions become coupled. It should be noted that Velcro type coupling means are known to those skilled in the art, and therefore are not necessary to discuss in further detail. The Velcro bands can be attached to first and second belt portions by typical means, such as stitching, adhesives, etc. The Velcro bands can have varying lengths and widths on the belt portions, such as 6 inches in length by 4 inches in height. It should be noted, however, that the Velcro type bands can be longer, wider, shorter or narrower (e.g., lengths of 4 inches, 8 inches, 10 inches, etc.; widths of 3 inches, 5 inches, 6 inches, etc.).

In another embodiment, the removable coupling means is a buckle (not shown) adapted to slide an end portion of first belt portion 310 and an end of second belt portion 320 for fastening the first and second belt portions together with the buckle. A typical buckle (not shown) can be used where the buckle is only attached to one belt portion (statically) and the other belt portion slides through one portion of the belt and over another portion of the buckle to tighten and lock. Such belt buckling devices are known to those skilled in the art and need not be discussed further.

In yet another embodiment the removable coupling means is at least one lace or string. In this embodiment first belt portion 310 and second belt portion 320 each have a plurality of through holes or eyelets. The lace or string is removably coupled through the plurality of through holes for fastening first belt portion 310 and second belt portion 320 together. That is, a typical lace up design can be implemented where the lace(s) are placed through the eyelets (through holes) and a knot or bow is made to tighten or vary the length of recovery belt apparatus 300.

As illustrated in FIG. 3A recovery belt apparatus 300 includes pad portion 330 coupled with belt portions 310 and 320 at end portions 305 and 307, respectively. This embodiment is such that pad portion 330 is adapted to be placed over an incision on a person's abdomen post surgery where the incision is substantially horizontal across a person's abdomen. It should be noted that this embodiment also is applicable to angled incisions up to a 45 degree slant.

As illustrated in FIG. 3B, one embodiment of recovery belt apparatus 350 has an alternate pad and belt structure for use when an incision is substantially vertical to a person's abdomen, e.g., an emergency C section incision (i.e., vertical incision). In this embodiment, pad portion 370 is adapted to be placed perpendicular to a substantially vertical incision on a person's abdomen. It should be noted that this embodiment also is applicable to angled incisions greater than a 45 degree slant (i.e., 45 to 90 degrees where the midsection of an abdomen can be considered the reference plane). In this embodiment, pad 370 is coupled with first belt portion 360 at a first side portion 308, and coupled with second belt portion 365 at second side portion 309. In this embodiment, first belt portion 360 has a first end 361 and a second end 362, where end 362 has a wider cross section than end 361 in order to couple pad 370 wide enough at first side portion 308. That is, first belt portion 360 has a trapezoidal shape. In this embodiment, second belt portion 365 has a first end 366 and a second end 367, where end 367 has a wider cross section than end 366 in order to couple pad 370 wide enough at second side portion 309. That is, second belt portion 365 has a trapezoidal shape. Similarly to the embodiments discussed relative to FIG. 3A, the embodiment in FIG. 3B has similar belt portion coupling means, and pad and belt materials. FIG. 4B illustrates a side view of the embodiment illustrated in FIG. 3B. FIG. 5B illustrates the embodiment illustrated in FIG. 3B attached to a person's abdomen.

In another embodiment, pad portions 300 and 370 has a fill opening (not shown). The fill opening has fastening means for closing the fill opening. The fill opening is to allow addition and removal of filler material (e.g., pillow filler such as foam, down, imitation down, poly-fill material, etc.). In these embodiments (i.e., substantially vertical and substantially horizontal post surgery applications: FIG. 3A and FIG. 3B) the fill opening allows fill material to be added and removed to adjust thickness of pad portions 330 and 370. The fastening means of the fill opening can be a zipper or Velcro type bands disposed on inner and outer side surfaces of the fill opening to allow opening and closing of the fill opening. In one embodiment, the fill opening is the whole length of pad portion 330 or 370. In other embodiments, the length of fill opening can be shorter, such as two inches from end 305 and 307).

In one embodiment recovery belt apparatus 300 and 350 further include a pad portion cover (not shown) where the cover is adapted to fit over the pad portion (i.e., pad portion 330 and pad portion 370). This cover is similar to a pillow case but adapted to snugly fit recovery belt apparatus 300 and 350. The pad portion cover allows replacement covers to be attached to the pad portions in case the cover gets soiled. Also, the pad portion covers can be various colors and have various designs in order to blend or match with a person's choice of clothing. Therefore, recovery belt apparatus 300 and 350 can be worn on the outside of a person's clothing or under a person's clothing. This can add aesthetic effect to wearing a post surgery belt apparatus, besides the functional purpose of adding comfort and reducing pain when sneezing, coughing, laughing, walking, etc.

It should also be pointed out that recovery belt apparatus 300 and 350 can be worn by females post pregnancy to assist in flattening out the abdominal area and adding additional comfort. The belt can be adjusted to put pressure on a female's abdominal area as required.

FIG. 3C illustrates a lumbar support pad. In one embodiment lumbar support pad 380 is adapted to be coupled to recovery belt apparatus 300 and 350. In this embodiment, lumbar support pad 380 has Velcro attached to rear portion 381. Also in this embodiment, first belt portion 360, second belt portion 365, or both belt portions have Velcro (reference 382) coupled to the inside (portion adjacent to a person's back when worn). Lumbar support pad 380 is placed on the inside portion of the belt portion adjacent to a person's back to add support. The Velcro applied to the belt portions and lumbar support pad 380 hold lumbar support pad 380 in place. In one embodiment, lumbar support pad 380 includes outside covering 383. Covering 383 is material such as terry cloth, nylon, cotton, etc. In one embodiment, covering 383 is removably coupled to lumbar support pad 380 to allow different styles, colors, allow covering 383 to be cleaned, etc.

In one embodiment, lumbar support pad 380 is made of foam disposed within covering 383. In another embodiment, lumbar support pad 380 is air filled through a valve (not shown) and air pressure is adjusted as desired. In another embodiment, lumbar support pad is filled with a gel, similar to the above-discussed pad 330 and 370. By adjusting belt portions and attaching lumbar support pad 380, a person wearing a recovery belt apparatus (i.e., 300, 350) gains lumbar support in addition to comfort from pad 330 or pad 370. FIG. 3D illustrates a vertical side view of the embodiment illustrated in FIG. 3C.

FIG. 6 illustrates an embodiment having two sets of belt portions to allow the belt apparatus to be used for substantially vertical and substantially horizontal post surgery recovery applications. Belt apparatus 600 includes pad portion 630, which includes first end 650, second end 655, third end 640 and fourth end 640. Belt apparatus 600 further includes first removable coupling means 611 for coupling first belt portion (610 or 650) to one of the pad portion's ends (i.e. 650 or 640, respectively), second removable coupling means 612 for coupling a second belt portion (620 or 625) to another of the pad portion's ends (i.e., 655 or 645, respectively), third removable coupling means (613 and 614) for coupling the first belt portion (i.e., 610 or 650) to the second belt portion (i.e., 620 or 625, respectively). Pad portion 630 is adapted to fit to a person's abdomen and the third removable coupling means (613 and 614) adjusts pressure of pad portion 630 on a person's abdomen.

Belt apparatus 600 is such that first removable coupling means 611, second removable coupling means 612, and third removable coupling means (613 and 614) are each a Velcro type band disposed on first pad end portion 650, second pad end portion 655, third pad end portion 640 and fourth pad end portion 645, first belt portion 610 or 650 and second belt portion 620 or 625 for fastening first and second belt portions to pad 630, and the first and second belt portions together in an overlapping manner. That is, each of the end portions of pad 630 have either a Velcro hook portion or loop portion. The belt end portions for attaching to pad 630's ends have the opposite (i.e., either hook or loop). Therefore, the belt ends can be attached to pad 630's ends in either a horizontal or vertical manner by using either belt portions 610 and 620, or belt portions 650 and 625, respectively.

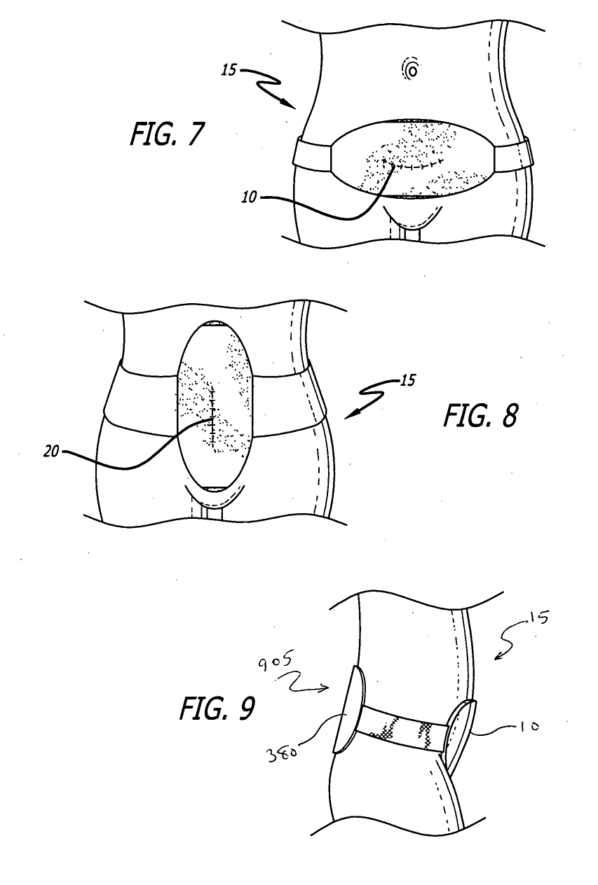

Similarly to the embodiments discussed above, belt apparatus 600 is made having similar features and of similar material. Also similar to the above-mentioned embodiments, third removable coupling means can be a buckle adapted to slide an end portion of one belt portion and an end of another belt portion for fastening first and second belt portions together with the buckle, or lace (or string) and eyelets (through holes). In one embodiment, pad portion 630 can be similar to embodiments mentioned above having a fill opening and using similar fill material and fill opening fastening means. In yet another embodiment, pad portion 630 can have a pad cover similar to the above-mentioned embodiments. FIG. 7 illustrates the embodiment shown in FIG. 6 being worn on a person's abdomen for a substantially horizontal surgical incision.

FIG. 8 illustrates the embodiment illustrated in FIG. 6 being worn on a person's abdomen for a substantially vertical surgical incision. FIG. 9 illustrates the embodiment illustrated in FIG. 6 being worn on a person's abdomen for a substantially horizontal surgical incision along with the embodiment illustrated in FIGS. 3C-D worn on a person's lumbar area (reference 905). It should be noted that the embodiment illustrated in FIGS. 3C-D can also be placed with an embodiment as illustrated in FIG. 8.

It should also be pointed out that recovery belt apparatus 300 and 350 can be worn by females post pregnancy to assist in flattening out the abdominal area and adding additional comfort. The belt can be adjusted to put pressure on a female's abdominal area as required.

In another embodiment, lumbar support pad 380 is removably coupled to belt apparatus 600. In this embodiment, lumbar support pad 380 has Velcro attached to rear portion 381. Also in this embodiment, first belt portion 610 (615), second belt portion 620 (625), or each belt portions have Velcro (reference 382) coupled to the inside of the belt portions (portion adjacent to a person's back when worn). Lumbar support pad 380 is placed on the inside portion of the belt portion adjacent to a person's back to add lumbar support. The Velcro applied to the belt portions and lumbar support pad 380 hold lumbar support pad 380 in place. In one embodiment, lumbar support pad 380 includes outside covering 383. Covering 383 is material such as terry cloth, nylon, cotton, etc. In one embodiment, covering 383 is removably coupled to lumbar support pad 380 to allow different styles, colors, patterns, covering 383 to be cleaned, replaced, etc.

In one embodiment, lumbar support pad 380 is made of foam disposed within covering 383. In another embodiment, lumbar support pad 380 is air filled through a valve (not shown) and air pressure is adjusted as desired. In another embodiment, lumbar support pad is filled with a gel, similar to the above-discussed pad 330, 370 and 630. By adjusting belt portions and attaching lumbar support pad 380, a person wearing recovery belt apparatus 600 gains lumbar support in addition to comfort from pad 630.

By using the above mentioned embodiments for post abdominal or thoracic area surgical procedure recovery a person does not have to carry a pillow around, which occupies the person's hands. This allows a person to have their hand's free for such activities as nursing a newborn, holding a newborn, use one's hands for other purposes (i.e. a cane, a walker, etc.) walk without having to hold a pillow, etc. The embodiments mentioned above provide continuous support to the abdomen or thoracic area after a surgical procedure and are particularly useful when sneezing, coughing, laughing or walking. The embodiments can be worn day and night as support is needed. Since the belt portions are adjustable, a custom fit is allowed for the above-mentioned embodiments. Further, a female recovering from giving birth can comfort her abdominal area and be assisted with flattening the abdominal area after giving birth. Moreover, by using a lumbar support pad in conjunction with the abdominal pads, a person benefits by having the additional lumbar support.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

1. An apparatus comprising:
a pad portion coupled to a first belt portion on a first end and a second belt portion on a second end; and
a removable coupling means for coupling the first belt portion with the second belt portion,
wherein the pad portion is adapted to be placed over one of a person's abdomen and thoracic area, and the removable coupling means adjusts pressure of the pad portion on one of a person's abdomen and thoracic area.

2. The apparatus of claim 1, wherein the pad portion is a pillow, the pillow having a top portion and a bottom portion.

3. The apparatus of claim 2, the bottom portion having a concave shape.

4. The apparatus of claim 1, wherein the pad portion is filled with gel, the pad portion having a top portion and a bottom portion.

5. The apparatus of claim 4, the bottom portion having a concave shape.

6. The apparatus of claim 1, wherein the removable coupling means is a Velcro type band disposed on inner and outer side surfaces of the first belt portion and the second belt portion for fastening the first and second belt portions together in an overlapping manner.

7. The apparatus of claim 1, wherein the removable coupling means is a buckle adapted to slide an end portion of one of the first belt portion and an end of the second belt portion for fastening the first and second belt portions together with the buckle.

8. The apparatus of claim 1, wherein the removable coupling means is at least one lace, the first belt portion and the second belt portion each having a plurality of through holes, wherein the at least one lace is removably coupled through the plurality of through holes for fastening the first and second belt portions together.

9. The apparatus of claim 1, the pad portion adapted to be placed substantially parallel to an incision on one of a person's abdomen and thoracic area.

10. The apparatus of claim 1, the pad portion adapted to be placed substantially perpendicular with an incision on one of a person's abdomen and thoracic area.

11. The apparatus of claim 1, the pad portion having a fill opening, the fill opening having fastening means for closing the fill opening, the fill opening to hold varying amount of filler to adjust thickness of the pad portion.

12. The apparatus of claim 11, the fastening means is one of a zipper and Velcro type bands disposed on inner and outer side surfaces of the fill opening for one of opening and closing the fill opening.

13. The apparatus of claim 1, further including a pad portion cover, the cover adapted to fit over the pad portion.

14. The apparatus of claim 1, further including a lumbar support pad removably coupled to an inside portion of one of the first belt portion and the second belt portion.

15. The apparatus of claim 14, further including a lumbar support pad cover removably coupled to the lumbar support pad.

16. The apparatus of claim 1, wherein the pad portion is adapted to be placed over an incision on one of a person's abdomen and thoracic area.

17. The apparatus of claim 1, wherein the pad portion is adapted to assist in flattening a person's abdominal area after giving birth.

18. An apparatus comprising:
a pad portion having a first end, a second end, a third end and a fourth end;
a first removable coupling means for coupling a first belt portion to one of the pad portion's ends,
a second removable coupling means for coupling a second belt portion to another of the pad portion's ends, and
a third removable coupling means for coupling the first belt portion to the second belt portion,
wherein the pad portion is adapted to be placed on one of a person's abdomen and thoracic area, and the third removable coupling means adjusts the pad portion pressure on one of a person's abdomen and thoracic area.

19. The apparatus of claim 18, wherein the pad portion is a pillow, the pillow having a top portion and a bottom portion.

20. The apparatus of claim 19, the bottom portion having a concave shape.

21. The apparatus of claim 18, wherein the pad portion is filled with gel, the pad portion having a top portion and a bottom portion.

22. The apparatus of claim 21, the bottom portion having a concave shape

23. The apparatus of claim 18, wherein the first removable coupling means, the second removable coupling means, and the third removable coupling means are each a Velcro type band disposed on the first pad end portion, the second pad end portion, the third pad end portion and the fourth pad end portion, the first belt portion and the second belt portion for fastening the first and second belt portions to the pad, and the first and second belt portions together in an overlapping manner.

24. The apparatus of claim 18, wherein the third removable coupling means is a buckle adapted to slide an end portion of the first belt portion and an end of the second portion for fastening the first and second belt portions together with the buckle.

25. The apparatus of claim 18, wherein the first, the second and the third removable coupling means comprise at least one lace each, the first belt portion, the second belt portion and the pad ends each having a plurality of through holes, wherein each lace is removably coupled through the plurality of through holes for fastening the first and second belt portions together, and the first and second belt portions each to one of the pad's ends.

26. The apparatus of claim 18, the pad portion adapted to be placed substantially parallel with an incision on one of a person's abdomen and thoracic area.

27. The apparatus of claim 18, the pad portion adapted to be placed substantially perpendicular with an incision on one of a person's abdomen and thoracic area.

28. The apparatus of claim 18, the pad portion having a fill opening, the fill opening having fastening means for closing the fill opening, the fill opening to hold varying amount of filler to adjust thickness of the pad portion.

29. The apparatus of claim 28, the fastening means is one of a zipper and Velcro type bands disposed on inner and outer side surfaces of the fill opening for one of opening and closing the fill opening.

30. The apparatus of claim 18, further including a pad portion cover, the cover adapted to fit over the pad portion.

31. The apparatus of claim 18, further including a lumbar support pad removably coupled to an inside portion of one of the first belt portion and the second belt portion.

32. The apparatus of claim 31, further including a lumbar support pad cover removably coupled to the lumbar support pad.

33. The apparatus of claim 18, wherein the pad portion is adapted to assist in flattening a person's abdominal area after giving birth.

34. An apparatus comprising:
a pad portion coupled to a first belt portion on a first end and a second belt portion on a second end;
a removable coupling means for coupling the first belt portion with the second belt portion, and
a lumbar support pad removably coupled to an inside portion of one of the first belt portion and the second belt portion,
wherein the pad portion is adapted to be placed over one of a person's abdomen and thoracic area, and the removable coupling means adjusts pressure of the pad portion on one of a person's abdomen and thoracic area.

35. An apparatus comprising:
a pad portion having a first end, a second end, a third end and a fourth end;
a first removable coupling means for coupling a first belt portion to one of the pad portion's ends,
a second removable coupling means for coupling a second belt portion to another of the pad portion's ends,
a third removable coupling means for coupling the first belt portion to the second belt portion, and
a lumbar support pad removably coupled to an inside portion of one of the first belt portion and the second belt portion,
wherein the pad portion is adapted to be placed on one of a person's abdomen and thoracic area, and the third removable coupling means adjusts the pad portion pressure on one of a person's abdomen and thoracic area.
